(19) United States
(12) Patent Application Publication
(10) Pub. No.: US 2002/0141599 A1
(43) Pub. Date: Oct. 3, 2002

(54) Active noise canceling headset and devices with selective noise suppression

(75) Inventors: Miroslav Trajkovic, Ossining, NY (US); Srinivas Gutta, Buchanan, NY (US); Eric Cohen-Solal, Ossining, NY (US)

Correspondence Address: Michael E. Marion, Corporate Patent Counsel, U.S. Philips Corporation, 580 White Plains Road, Tarrytown, NY, 10591, US

(73) Assignee: Philips Electronics North America Corp.

(21) Appl. No.: 09/825,045

(22) Filed: Apr. 3, 2001

(51) Int. Cl. A61F 11/06; G10K 11/16; H03B 29/00

(52) U.S. Cl. 381/71.6; 381/71.14; 381/72

(57) ABSTRACT

A selective noise canceling device is disclosed that evaluates an external audio signal and determines whether a given external audio signal should be suppressed or reproduced. A selective noise suppression circuit processes the external audio signal to classify the external audio signal as noise to be suppressed or as a desired audio signal to be reproduced. An external audio signal, or a desired portion thereof, is reproduced if a portion of the external audio signal is likely to be of interest to the user. The desired external signal may optionally be amplified over the primary selected audio signal for emphasis and the selected audio signal may optionally be suppressed entirely when the desired external signal is reproduced.

### FIELD OF THE INVENTION

The present invention relates to active noise reduction techniques, and more particularly, to headsets and other devices employing such active noise reduction techniques.

### BACKGROUND OF THE INVENTION

Portable telephones and electronic devices are increasingly popular in today's mobile society. Cellular and wireless telephones, for example, receive a signal transmitted from a base station or base unit, respectively. The portable telephones allow users to make and receive telephone calls within a given radius of the base station (or base unit). Similarly, portable electronic devices, such as the Sony Walkman™, commercially available from Sony Corporation, including personal radios, cassette tape players, digital audio tape (DAT) players, MP3 players and compact disc players, allow a user to listen to desired audio content at virtually any location.

Such portable devices provide users with great flexibility and convenience. The portable nature of such devices, however, encourages users to utilize such devices from virtually any location, or even while traveling. Thus, users may be distracted when adjusting settings of the portable devices, such as when making a telephone call, or by the content itself, thereby potentially exposing the user to undue risks. In response to an increased number of motor vehicle accidents due to drivers distracted while making a telephone call, for example, a number of jurisdictions have enacted legislation requiring users of cellular telephones to employ hands-free cellular devices.

Portable telephones and electronic devices typically include headphones or integrated speakers to reproduce the audio signal. Headsets typically include a pair of earpieces coupled by a flexible headband that presses the earpieces against the head of the user. The application of the earpieces to the user's ears serves to reduce ambient noise in a passive manner. In many environments, however, such as on an airplane, passive noise reduction techniques are insufficient.

Thus, a number of manufacturers provide headsets that incorporate an active noise reduction feature for canceling noise signals. Bose Corporation of Framingham, Mass., for example, sells a line of aviation headsets for use by airline passengers. The aviation headsets reduce engine and wind noise, and provide improved reproduction of a selected audio signal. Generally, the Bose aviation headset employs a microphone to monitor external sound at the user's ear. The measured sound is compared with the selected audio signal that the user desires to hear. The difference between the measured sound and the selected audio signal is unwanted external noise. An opposing correction signal (antinoise) is then generated by the headset. The antinoise is an equal and opposite vibration, 180 degrees out of phase with the noise to be blocked. When the unwanted noise and generated antinoise collide, destructive interference effects operate to cancel each other out.

Such noise canceling headsets serve to cancel all noise, however, other than the selected audio signal. Generally, the noise cancellation feature emphasizes the selected audio signal, and suppresses all other audio signals. Thus, users of such headsets are in a relatively closed audio environment, essentially unaware of any outside audio stimulus other than associated with the selected audio signal. If another person speaks to a user of such headsets, for example, the noise cancellation feature would operate to suppress the speaker. Likewise, the noise cancellation feature would operate to suppress other audio signals, such as an emergency announcement or alarm, that would otherwise alert the user to a risk.

A need therefore exists for a noise canceling device that evaluates the content of an external audio signal and determines whether a given audio signal should be suppressed or reproduced. A further need exists for a noise canceling headset that reproduces external audio signals that are of interest to a user. Yet another need exists for a noise canceling headset that classifies an external audio signal based on its content.

### SUMMARY OF THE INVENTION

Generally, a selective noise canceling device is disclosed that evaluates an external audio signal and determines whether a given external audio signal should be suppressed or reproduced. The disclosed selective noise canceling device includes a selective noise suppression circuit that processes the external audio signal to classify the external audio signal as either noise to be suppressed or a desired audio signal to be reproduced. The portion of the external audio signal that is to be reproduced in accordance with the present invention is referred to herein as the “desired external signal.”

The present invention reproduces an external audio signal, or a desired portion thereof, if a portion of the external audio signal is likely to be of interest to the user. For example, the selective noise canceling device of the present invention may reproduce audio signals alerting the user to harm, as well as other audio signals directed to the user that are acoustically distinct from the general background noise of the environment.

The desired external signal may optionally be amplified over the primary selected audio signal for emphasis. In addition, the selected audio signal may optionally be suppressed entirely when the desired external signal is reproduced.

A more complete understanding of the present invention, as well as further features and advantages of the present invention, will be obtained by reference to the following detailed description and drawings.

### BRIEF DESCRIPTION OF THE DRAWINGS

FIG. 1 illustrates a selective noise canceling headset in accordance with the present invention;

FIG. 2 is a schematic block diagram illustrating the selective noise canceling headset of FIG. 1 in further detail;

FIG. 3 is a schematic block diagram illustrating a first embodiment of the selective noise suppression circuit of FIG. 2;

FIG. 4 is a schematic block diagram illustrating a second embodiment of the selective noise suppression circuit of FIG. 2; and

FIG. 5 is a schematic block diagram illustrating a third embodiment of the selective noise suppression circuit of FIG. 2.

### DETAILED DESCRIPTION

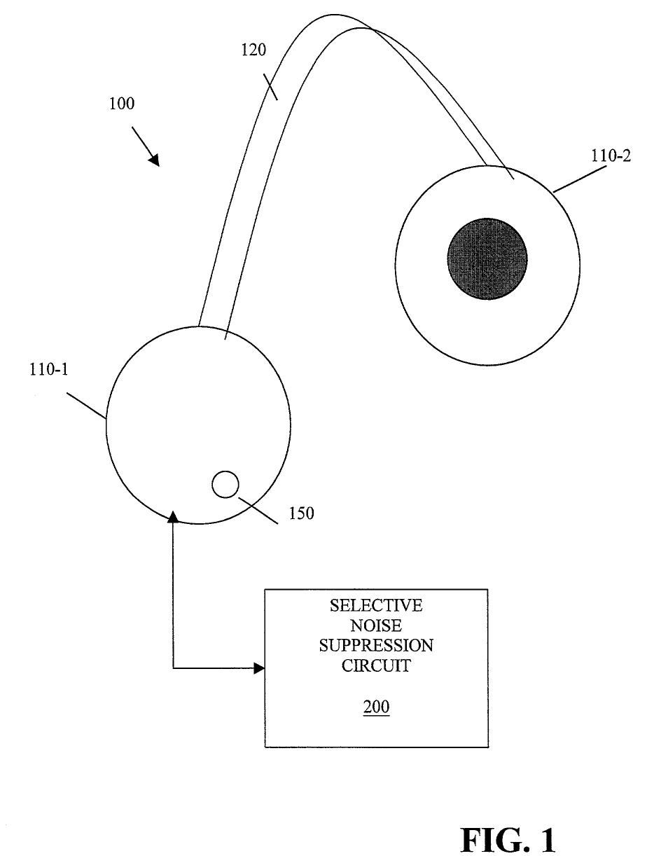

FIG. 1 illustrates a selective noise canceling headset 100 in accordance with the present invention. As shown in FIG. 1, the selective noise canceling headset 100 includes a pair of earpieces 110-1, 110-2 (collectively, earpieces 110) coupled by a flexible headband 120 that presses the earpieces 110 against the head of the user, in known manner. In addition, the selective noise canceling headset 100 includes one or more microphones 150 mounted on the earpieces 110 to monitor the external sound at the user's ear and generate a signal referred to herein as the “external audio signal.”

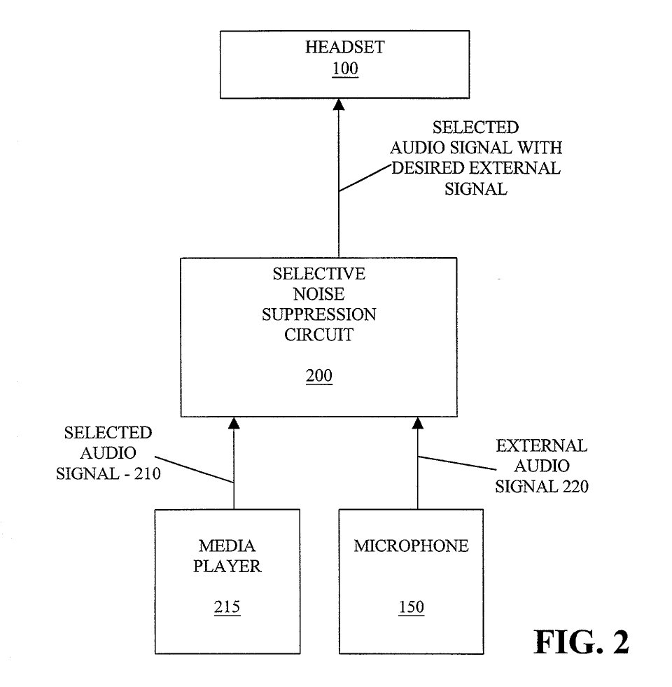

According to one feature of the present invention, the selective noise canceling headset 100 also includes a selective noise suppression circuit 200, discussed below in conjunction with FIG. 2, that processes the external audio signal obtained by the microphone 150 and selectively classifies the external audio signal as either noise to be suppressed or an audio signal to be reproduced. The portion of the external audio signal that is to be reproduced in accordance with the present invention is referred to herein as the “desired external signal.”

Generally, an external audio signal will be reproduced by the selective noise canceling headset 100 in accordance with the present invention if the audio signal is likely to be of interest to the user, as discussed further below. For example, the selective noise canceling headset 100 may reproduce audio signals alerting the user to harm, such a dog barking, an alarm or an automobile horn, and other audio signals directed to the user that are acoustically distinct from the general background noise of the environment, such as spoken words. The user can optionally specify the kinds of external audio noise to be reproduced in a given environment.

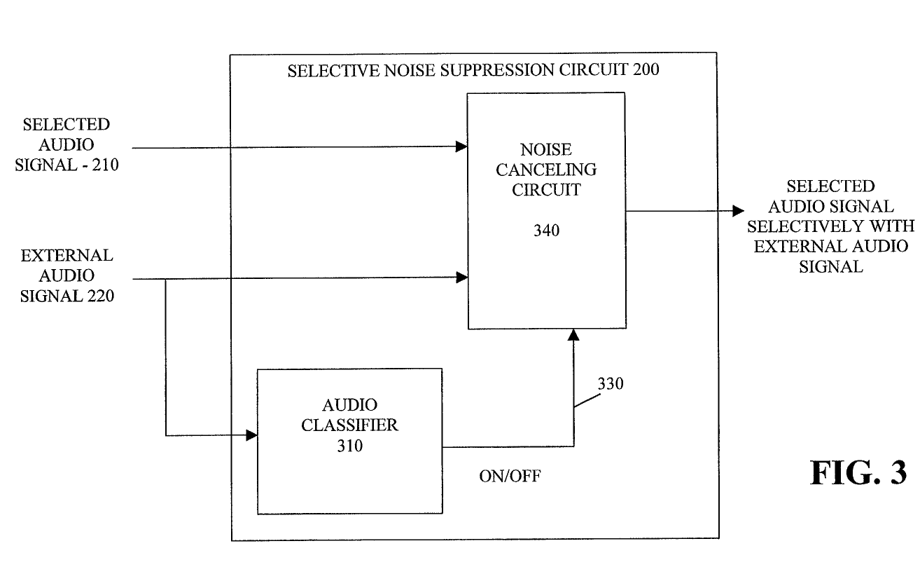

Thus, as shown in FIG. 2, the selective noise suppression circuit 200 receives the selected audio signal 210 from a media player 215, such as a personal radio, cassette tape player, DAT player, MP3 player, compact disc player or portable telephone, and the external audio signal 220 generated by the microphone 150. Various embodiments of the selective noise suppression circuit 200 are discussed below in conjunction with FIG. 3 through 5. Generally, the selective noise suppression circuit 200 processes the selected audio signal 210 and external audio signal 220 to reproduce the selected audio signal 210, as well as the desired external signal portion of the external audio signal 220, and suppress the remaining portions of the external audio signal 220 (noise).

Thus, the selective noise suppression circuit 200 applies the selected audio signal 210 and the desired external signal to the selective noise canceling headset 100. In a further variation, the selective noise suppression circuit 200 can optionally cancel the selected audio signal 210 as well and reproduce only the desired external signal, for example, in presence of a known threat.

FIG. 3 illustrates a first exemplary embodiment of the selective noise suppression circuit 200 of FIG. 2. As shown in FIG. 3, the selective noise suppression circuit 200 includes an audio classifier 310 and a noise canceling circuit 340. As shown in FIG. 3, the audio classifier 310 processes the external audio signal and returns a binary zero (0) if the external audio signal is noise and should be cancelled by the noise canceling circuit 340, or a binary one (1) if the external audio signal is useful and should be reproduced as the desired external signal with the selected audio signal. In this manner, the audio classifier 310 indicates whether the entire external audio signal should be suppressed or reproduced by the noise canceling circuit 340. The audio classifier 310 may be embodied in accordance with the audio content analysis techniques described in, for example, Silvia Pfeiffer et al., “Automatic Audio Content Analysis,” Proc. ACM Multimedia 96, 21-30, Boston, Mass. (November 1996), incorporated by reference herein.

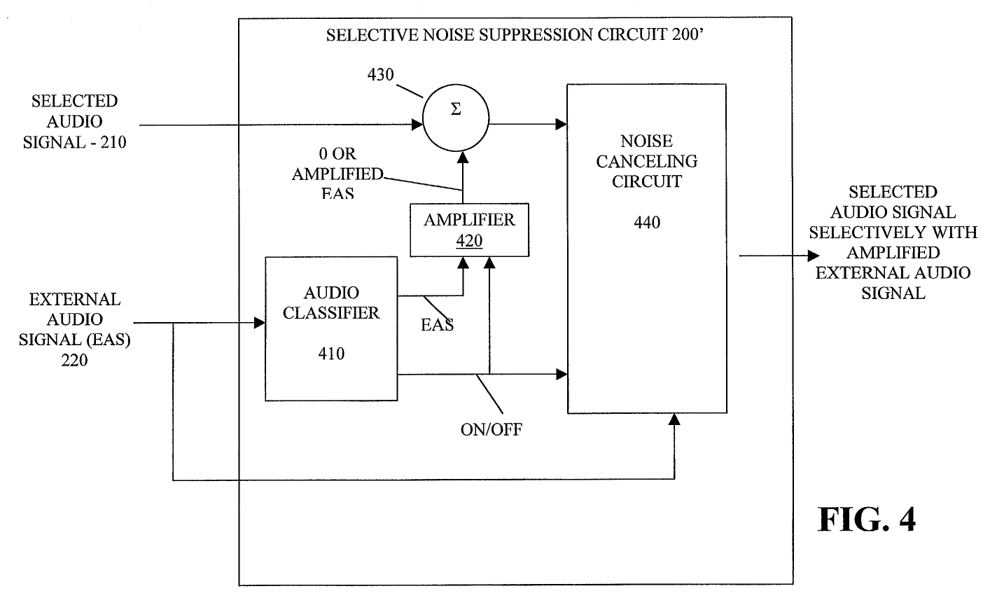

FIG. 4 illustrates a second exemplary embodiment of the selective noise suppression circuit 200′ of FIG. 2. As shown in FIG. 4, the selective noise suppression circuit 200′ includes an audio classifier 410, an amplifier 420, an adder 430 and a noise canceling circuit 440. The audio classifier 410 and noise canceling circuit 440 can be embodied in the same manner as the corresponding elements in FIG. 3. Thus, the audio classifier 410 processes the external audio signal and returns a binary zero (0) or one (1) if the external audio signal is noise and should be cancelled or if the external audio signal is useful and should be reproduced, respectively.

In addition, if the external audio signal is useful, the signal is amplified by an amplifier 420 also controlled by the on/off signal generated by the audio classifier 410. Thus, the output of the amplifier 420 is zero (0) if the external audio signal is not useful, or an amplified version of the external audio signal if the external audio signal is useful. The output of the amplifier 420 is added to the selected audio signal by an adder 430, and the combined output is applied to the noise canceling circuit 440. In this manner, the output of the noise canceling circuit 440 can emphasize the amplified external audio signal over the selected audio signal, if desired.

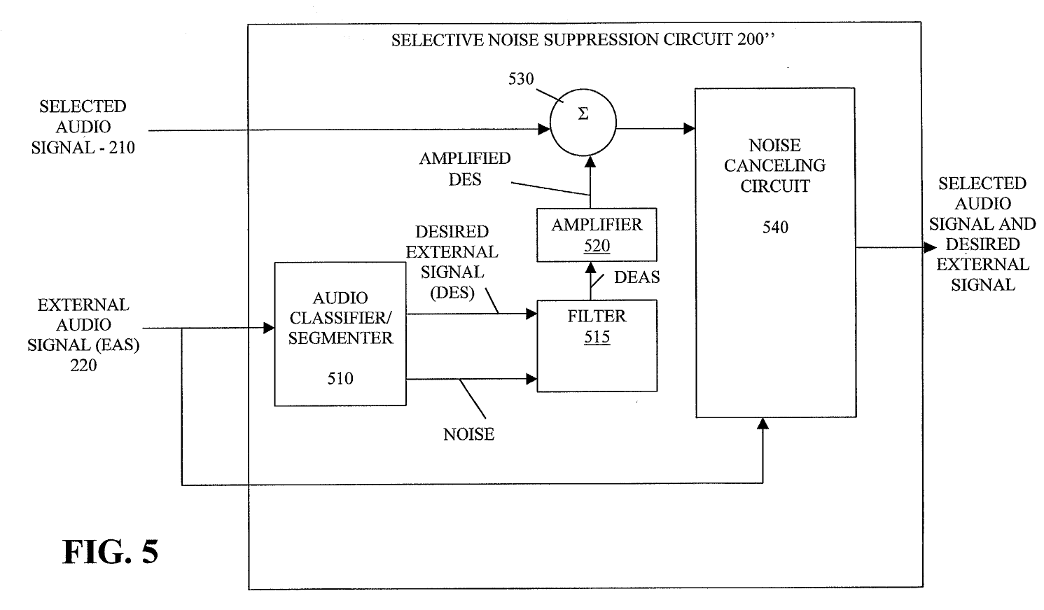

FIG. 5 illustrates a third exemplary embodiment of the selective noise suppression circuit 200″ of FIG. 2. As shown in FIG. 4, the selective noise suppression circuit 200″ includes an audio classifier/segmenter 510, an amplifier 520, an adder 530 and a noise canceling circuit 540. The audio classifier 510, adder 530 and noise canceling circuit 540 can be embodied in the same manner as the corresponding elements in FIGS. 3 and 4. In the embodiment of FIG. 5, the amplifier 520 is no longer controlled by the output of the audio classifier 510, but rather amplifies any signal present at its input.

The audio classifier/segmenter 510 segments the external audio signal into various signals, classifies each individual signal as useful or noise, and suppresses the noise using a filter 515. The output of the filter 515 is the desired external signal (DES) . The audio classifier/segmenter 510 may be embodied in accordance with the techniques described in T. Zhang and C-C. Jay Kuo, “Heuristic Approach for Generic Audio Data Segmentation and Annotation,” Proc. ACM Multimedia 99 (ACM Special Interest Groups), Nov. 5, 1999, incorporated by reference herein. Generally, the audio classifier/segmenter 510 will reproduce the external audio signal if the audio signal is likely to be of interest to the user. For example, the selective noise canceling headset 100 may reproduce audio signals alerting the user to harm, such an alarm or an automobile horn, and other audio signals directed to the user that are acoustically distinct from the general background noise of the environment, such as spoken words. The desired external signal is amplified by the amplifier 520 and the output of the amplifier 520 is added to the selected audio signal by an adder 530. The combined output is applied to the noise canceling circuit 540. In this manner, the output of the noise canceling circuit 540 can emphasize the amplified desired external signal, if desired, over the selected audio signal.

In a further variation, the audio classifier/segmenter 510 can initiate the replay of a recorded message associated with a given predefined audio segment. For example, the audio classifier/segmenter 510 can initiate a message such as “dog barking on left side” if such a signal is identified in accordance with the present invention. In an embodiment where the external audio signal is amplified, the final audio signal can be defined as follows:

Final Audio Signal=α*(external audio signal)+(1-α)*(selected audio signal),

where a determines the intensity of the signal.

It is to be understood that the embodiments and variations shown and described herein are merely illustrative of the principles of this invention and that various modifications may be implemented by those skilled in the art without departing from the scope and spirit of the invention.

What is claimed is:

1. A selective noise canceling headset, comprising:
at least one earpiece for reproducing a selected audio signal;
a microphone for monitoring an external audio signal in a vicinity of said headset; and
a selective noise suppression circuit for analyzing said external audio signal and for selectively reproducing at least a portion of said external audio signal that is likely to be of interest to a user.

2. The selective noise canceling headset of claim 1, wherein said reproduced portion of said external audio signal is an alarm audio signal.

3. The selective noise canceling headset of claim 1, wherein said reproduced portion of said external audio signal is acoustically distinct from a general background noise of a local environment.

4. The selective noise canceling headset of claim 1, wherein said reproduced portion of said external audio signal is associated with speech directed to a user of said headset.

5. The selective noise canceling headset of claim 1, wherein said selective noise suppression circuit suppresses said external audio signal unless a portion of said external audio signal is likely to be of interest to a user.

6. The selective noise canceling headset of claim 1, wherein said selective noise suppression circuit segments said external audio signal and reproduces only a desired portion of said external audio signal that is likely to be of interest to a user.

7. The selective noise canceling headset of claim 1, wherein said selective noise suppression circuit emphasizes a desired portion of said external audio signal that is likely to be of interest to a user over said selected audio signal.

8. A selective noise canceling device, comprising:
a microphone for monitoring an external audio signal; and
a selective noise suppression circuit for analyzing said external audio signal and for selectively reproducing at least a portion of said external audio signal that is likely to be of interest to a user.

9. The selective noise canceling device of claim 8, wherein said reproduced portion of said external audio signal is an alarm audio signal.

10. The selective noise canceling device of claim 8, wherein said reproduced portion of said external audio signal is acoustically distinct from a general background noise of a local environment.

11. The selective noise canceling device of claim 8, wherein said reproduced portion of said external audio signal is associated with speech directed to a user of said device.

12. The selective noise canceling device of claim 8, wherein said selective noise suppression circuit suppresses said external audio signal unless a portion of said external audio signal is likely to be of interest to a user.

13. The selective noise canceling device of claim 8, wherein said selective noise suppression circuit segments said external audio signal and reproduces only a desired portion of said external audio signal that is likely to be of interest to a user.

14. The selective noise canceling device of claim 8, wherein said selective noise suppression circuit emphasizes a desired portion of said external audio signal that is likely to be of interest to a user over a selected audio signal.

15. A selective noise canceling method, comprising:
monitoring an external audio signal;
analyzing said external audio signal; and
selectively reproducing at least a portion of said external audio signal that is likely to be of interest to a user.

16. The selective noise canceling method of claim 15, wherein said reproduced portion of said external audio signal is an alarm audio signal.

17. The selective noise canceling method of claim 15, wherein said reproduced portion of said external audio signal is acoustically distinct from a general background selective noise of a local environment.

18. The selective noise canceling method of claim 15, wherein said reproduced portion of said external audio signal is associated with speech directed to a user of said method.

19. The selective noise canceling method of claim 15, further comprising the step of suppressing said external audio signal unless a portion of said external audio signal is likely to be of interest to a user.

20. The selective noise canceling method of claim 15, further comprising the step of segmenting said external audio signal and reproducing only a desired portion of said external audio signal that is likely to be of interest to a user.

21. The selective noise canceling method of claim 15, further comprising the step of emphasizing a desired portion of said external audio signal that is likely to be of interest to a user over said selected audio signal.
